(19) United States
(12) Patent Application Publication
(10) Pub. No.: US 2004/0067965 A1
(43) Pub. Date: Apr. 8, 2004

(54) Antiviral method of use

(76) Inventors: Douglas Wayne Balogh, Carmel, IN (US); Joseph Matthew Colacino, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/470,391

(22) PCT Filed: Feb. 4, 2002
(86) PCT No.: PCT/US02/01233

(30) Foreign Application Priority Data
Feb. 16, 2001 (US) 60269486
May 24, 2001 (US) 60293297

(51) Int. Cl. A61K 31/519

(52) U.S. Cl. 514/262.1

(57) ABSTRACT

The present invention provides a method for treating HCMV in a human requiring said treatment, which comprises administering to said human an effective amount of ALIMTA.

Human cytomegalovirus (HCMV) is a herpesvirus commonly found in about 50% of the general population. About 90% of people with HIV carry HCMV. In the general population, the virus usually remains latent in the tissues of the body after the initial infection. It can, however, be shed in the mouth, urine, and genital tract, serving as a source of infection for other people. Infection with HCMV can result in secondary, more severe infections if the immune system becomes compromised for any reason.

Symptoms primarily depend on the age of the person and how strong their immune system is. HCMV may infect a healthy baby in utero. Roughly 5% of infants who get HCMV through vertical transmission have serious birth defects. These can include brain damage, growth failure, blindness, and other defects. This problem usually occurs when the mother becomes infected with HCMV for the first time during pregnancy. When HCMV causes infection in early childhood, it usually results in no symptoms at all. This is thought to be the most common form of HCMV infection.

During the teenage and young adult years, infection with HCMV can cause a syndrome similar to that of infectious mononucleosis, or “mono.” Here the symptoms include sore throat, fatigue, fevers, and swollen glands and can last for weeks or even months. Full recovery generally occurs without treatment.

In the general adult population, HCMV lies dormant, but may be associated with the development of coronary artery disease. Infection with HCMV has been associated with the development of arterial plaques and atherosclerosis.

HCMV can cause serious problems in people with weakened immune systems. This is most commonly a problem in people with AIDS or in those patients on immunosuppressive therapy. HCMV infects between 75 and 100% of HIV positive patients. The most common complications associated with HCMV include chorioretinitis; gastrointestinal tract infections, including hepatitis, esophagitis, colitis, gastritis, and pancreatitis; neurologic involvement, including encephalitis and polyradiculitis; pulmonary involvement; and epididymitis.

People with widespread cancer or people who receive organ or bone marrow transplants are commonly affected. Infection may be due to a first time exposure to HCMV or as a result of reactivated HCMV. In transplant and cancer patients, HCMV usually causes pneumonia or a gastrointestinal infection resulting in diarrhea, which may cause death. Furthermore, HCMV contributes to the development of chronic allograft dysfunction in solid organ transplant recipients. The relationship between HCMV disease and the development of bronchiolitis obliterans in lung transplant recipients is well established. Additionally, HCMV is one of a number of risk factors that may lead to allograft injury. Direct viral invasion of the allograph may cause HCMV hepatitis in liver or kidney transplant patients. In addition to direct syndromes produced by HCMV, infection with this virus may increase the risk for fungal and other opportunistic infections, such as Pneumocystis carinii pneumonia and Epstein-Barr virus-related posttransplant lymphoproliferative disease.

Most people have been infected with HCMV by the time they are adults. Anyone receiving a blood transfusion or an organ transplant is at risk for a HCMV infection. Furthermore, people with weakened immune systems and unborn children are at risk for severe disease.

Treating active HCMV in people with weakened immune systems is done with antiviral agents, such as ganciclovir, foscarnet, and cidofovir. These drugs may have side effects that include granulocytopenia and anemia for ganciclovir; nephrotoxicity, neurotoxicity, and electrolyte disturbance for foscarnet; and nephrotoxicity, neurotoxicity, and alopecia for cidofovir. Recently, Tomudex® has been found to be active against HCMV. Tomudex® has side effects that consist of rash, diarrhea, decreased production of blood cells by the bone marrow, pyrexia, nausea and vomiting, and loss of appetite. Despite a number of therapeutic approaches, HCMV remains a significant cause of morbidity and mortality in immunosuppressed patients.

Pyrrolo[2,3-d]pyrimidine based antifolates have been used for a number of years as chemotherapeutic agents in the treatment of cancer. A number of such pyrrolo[2,3-d]pyrimidine based antifolates are known (see: for example, U.S. Pat. Nos. 4,997,838; 5,106,974; 5,939,420; and 5,877,178, incorporated by reference herein). Disodium N-[4-[2-(2-amino-3,4-dihydro-4-oxo-7H-pyrrolo[2,3-d]pyrimidin-5-yl)ethyl]benzoyl]-L-glutamic acid salt, also known as ALIMTA, is one such compound. ALIMTA is currently in clinical trials for use as an anticancer treatment in patients exhibiting a wide variety of solid tumors. Extensive research and evaluation has revealed that ALIMTA is a potent inhibitor of several folate-requiring enzymes, including thymidylate synthase, dihydrofolate reductase and glycinamide ribonucleotide formyltransferase.

Surprisingly and unexpectedly, we have now found that ALIMTA is useful as an anticytomegalovirus agent.

The present invention provides a method for treating HCMV in a human requiring said treatment, which comprises administering to said human an effective amount of ALIMTA.

The present invention further provides the use of ALIMTA for the manufacture of a medicament for the treatment of HCMV.

Additionally, the present invention contemplates a therapeutic package for dispensing to, or for use in dispensing to, a patient being treated for HCMV comprising one or more unit doses, each unit dose comprising an amount of ALIMTA therein such that periodic administration of one or more of said unit doses is effective to treat HCMV; and a finished pharmaceutical container therefor, said container further containing or comprising labeling, said labeling indicating that ALIMTA is indicated for the treatment of patients with HCMV.

Additionally, the present invention contemplates an article of manufacture comprising packaging material and ALIMTA contained within said packaging material, wherein ALIMTA is therapeutically effective for treating HCMV, and wherein the packaging material comprises a label which indicates that ALIMTA can be used to treat HCMV.

ALIMTA, disodium N-[4-[2-(2-amino-4,7-dihydro-4-oxo-3H-pyrrolo[2,3-d]-pyrimidin-5-yl)ethyl]benzoyl]-L-glutamic acid salt, is represented by formula I:

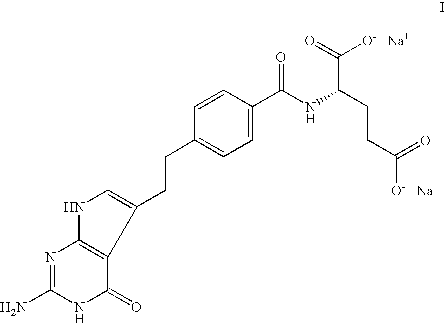

The compound of formula I can exist in tautomeric equilibrium with the corresponding 4(3H)-oxo compound. For illustrative purposes, the equilibrium for the pyrrolopyrimidine ring system and the numbering thereof, are shown below:

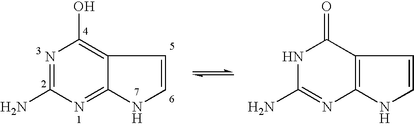

For convenience, the 4(3H)-oxo form is depicted in formula I, and the corresponding nomenclature is used throughout this specification. However, it is understood that such depictions include the corresponding tautomeric 4-hydroxy form.

ALIMTA was first taught by U.S. Pat. No. 5,344,932, the teachings of which are herein incorporated by reference in its entirety.

In the method according to the invention, the terms “treat” or “treating” bear their usual meaning which includes preventing, prohibiting, alleviating, ameliorating, halting, restraining, slowing or reversing the progression, or reducing the severity of HCMV.

In the method according to the invention, the term “HCMV” refers to human cytomegalovirus. The human cytomegalovirus is a herpesvirus that causes cellular enlargement and formation of eosinophilic inclusion bodies.

In the method according to the invention, the term “effective amount” refers to an amount of a compound or drug, which is capable of performing the intended result. For example, an effective amount of ALIMTA that is administered in an effort to treat HCMV is that amount which is required to prevent, prohibit, alleviate, ameliorate, halt, restrain, slow or reverse the progression, or reduce the severity of HCMV.

The present invention also includes methods employing pharmaceutical formulations, which contain, as the active ingredient, ALIMTA, in association with pharmaceutical carriers. A skilled artisan would know of such formulations and their manufacture, see, e.g., REMINGTON'S PHARMACEUTICAL SCIENCES, (16th ed. 1980).

The formulations are preferably formulated in a unit dosage form, each dosage containing from about 0.1 to about 100 mg, more usually about 10 to about 30 mg, of the active ingredient. The term “unit dosage form” refers to physically discrete units suitable as unitary dosages for human subjects, each unit containing a predetermined quantity of active material calculated to produce the desired therapeutic effect, in association with a suitable pharmaceutical excipient.

ALIMTA is effective over a wide dosage range. For example, dosages per day normally fall within the range of about 0.5 to about 30 mg/kg of body weight. However, it will be understood that the amount of ALIMTA actually administered will be determined by a physician, in the light of the relevant circumstances, including the condition to be treated, the chosen route of administration, the age, weight, and response of the individual patient, and the severity of the patient's symptoms, and therefore the above dosage ranges are not intended to limit the scope of the invention in any way. In some instances dosage levels below the lower limit of the aforesaid range may be more than adequate, while in other cases still larger doses may be employed without causing any harmful side effect, provided that such larger doses are first divided into several smaller doses for administration throughout the day.

ALIMTA can be administered in the form of a pharmaceutical composition in combination with pharmaceutically acceptable carriers or excipients, the proportion and nature of which are determined by the solubility and chemical properties of the compound in the carriers and/or excipients selected, the chosen route of administration, and standard pharmaceutical practice.

Pharmaceutical compositions are prepared in a manner well known in the pharmaceutical art see, e.g., REMINGTON'S PHARMACEUTICAL SCIENCES, (16th ed. 1980). The carrier or excipient may be a solid, semi-solid, or liquid material, which can serve as a vehicle or medium for the active ingredient. Suitable carriers or excipients are well known in the art. The pharmaceutical composition may be adapted for oral, inhalation, parenteral, or topical use and may be administered to the patient in the form of tablets, capsules, aerosols, inhalants, suppositories, solution, suspensions, or the like.

The following formulation example is illustrative only and is not intended to limit the scope of the invention in any way. “Active ingredient” means ALIMTA.

### EXAMPLE 1

| | |
|---|---|
| active ingredient | 4% (total solution) |
| L-cysteine | 0.03% (total solution) |
| a pharmaceutically acceptable excipient | water |

The pH of the solution was adjusted to 8.5 using sodium hydroxide. The pH-adjusted solution was protected from light. The solution was purged with nitrogen for twenty minutes and then sterile filtered. The formulation was dispensed into prewashed, depyrogenated vials and then stoppered with a prewashed, presterilized Teflon coated stopper. Caps were attached using a crimper. The sterile filtration and dispensing steps were conducted using a nitrogen isolator (5% v/v Oxygen).

### Utility Test Methods

The unexpected activity of ALIMTA as an anticytomegalovirus can be evidenced by tests conducted in human foreskin or fetal lung cells. All materials required for this testing are commercially available. The testing method described is well documented in the literature, see, e.g. Colacino, J M and Lopez C., 1983, “Efficacy and selectivity of some nucleoside analogs as anti-human cytomegalovirus agents.” Antimicrob. Agents Chemother. 24:505-508; Colacino, J M and Lopez C., 1985, “Antiviral activity of 2′-deoxy-2′-fluoro-beta-D-arabinofuranosyl-5-iodocytosine against human cytomegalovirus in human skin fibroblasts.” Antimicrob. Agents Chemother. 28:252-258; and Mauldin, S C, Paget C J Jr., Jones C D, Colacino J M, Baxter A J, Staschke K A, Johansson N G, and Vrang L., 1998, “Synthesis and antiviral activity of prodrugs of the nucleoside 1-[2′,3′-dideoxy-3′-C-(hydroxymethyl)-beta-D-erythropentofuranosyl] cytosine.” Bioorg. Med Chem. 6:577-585.

ALIMTA was prepared by methods well known in the art, see e.g. C. J. Barnett, et al., 1999, “A Practical Synthesis of Multitargeted Antifolate LY231514.” Org. Process Res. and Dev., 3, 184-188. A stock suspension of ALIMTA is then prepared in phosphate buffered saline and diluted as appropriate.

### Cell Culture

An accepted procedure for detecting activity of different classes of drugs against HCMV for which there is a good correlation with the treatment of HCMV in humans is the inhibition of virus replication in cell cultures and comparing virus replication in the presence of ALIMTA to virus replication in the absence of ALIMTA.

Human fibroblast cells, foreskin or fetal lung cells, are infected with human cytomegalovirus. The cells are incubated with standard cell culture media containing no or increasing concentrations of ALIMTA. After a period of time, the number of plaques observed in the non-treated infected cell control cultures is compared to the number of plaques observed in the treated infected cell cultures. Additionally the yield of virus produced in the non-treated infected cell control cultures is compared to the yield of virus produced in the treated infected cell cultures.

The percent inhibition of virus replication can be determined and the concentration of molecule required to inhibit viral replication by 50% (IC50) and 90% (IC90) can be calculated using linear regression analysis. Percent inhibition of 50 or higher at concentrations, which are not cytotoxic denotes selective antiviral activity and indicates that the drug is useful for the treatment of infections caused by HCMV. ALIMTA had an inhibition of greater than 50% at a concentration of 0.29 μM (IC50) and inhibited cell replication by 50% at a concentration of >100 μM (TC50) yielding a selective index (TC50/IC50) of >344.8.

Results:

HCMV SCREENING SUMMARY

| | |
|---|---|
| Compound | Results |

| | | | | |
|---|---|---|---|---|
| Compound | IC50 | TC50 | TI | Comments |

| | | | | |
|---|---|---|---|---|
| ALIMTA | 0.29 μM | >100 μM | >344.8 | High activity |
| Ganciclovir | 6.37 μM | >100 μM | >15.7 | Control |

IC50 = 50% Inhibitory Concentration of HCMV strain AD169 induced plaque formation in MRC-5 Cells.
TC50 = 50% Cytotoxic Concentration as measured in MRC-5 Cells with Promega's Cell Titer 96 Aqueous One Solution
TI (Therapeutic Index) = TC50/IC50

We claim:

1. A method for treating HCMV in a human requiring said treatment, which comprises administering to said human an effective amount of ALIMTA.

2. A use of ALIMTA for the manufacture of a medicament for the treatment of HCMV.

3. A therapeutic package for dispensing to, or for use in dispensing to, a patient being treated for HCMV comprising one or more unit doses, each unit dose comprising an amount of ALIMTA therein such that periodic administration of one or more of said unit doses is effective to treat HCMV; and a finished pharmaceutical container therefor, said container further containing or comprising labeling, said labeling indicating that ALIMTA is indicated for the treatment of patients with HCMV.

4. An article of manufacture comprising packaging material and ALIMTA contained within said packaging material, wherein ALIMTA is therapeutically effective for treating HCMV, and wherein the packaging material comprises a label which indicates that ALIMTA can be used to treat HCMV.
